(19) United States
(12) Patent Application Publication
(10) Pub. No.: US 2006/0205080 A1
(43) Pub. Date: Sep. 14, 2006

(54) Formulations for therapeutic viruses having enhanced storage stability

(76) Inventors: David Frey, Half Moon Bay, CA (US); Cassandra Shu, Castro Valley, CA (US)

Correspondence Address: DLA Piper Rudnick Gray Cary LLP, Suite 800, 153 Townsend Street, San Francisco, CA, 94107-1957, US

(21) Appl. No.: 11/363,022

(22) Filed: Feb. 27, 2006

Related U.S. Application Data
(60) Provisional application No. 60/656,883, filed on Mar. 1, 2005.

(51) Int. Cl. C12N 15/861

(52) U.S. Cl. 435/456

(57) ABSTRACT

Therapeutic viral formulations having enhanced storage stability are described. The formulations comprise a viral vector in addition to one or more of an aqueous cosolvent, a reversible viral-encoded protease inhibitor and a mild reducing agent or other agent that prevents specific degradation of viral components.

### CROSS-REFERENCES TO RELATED APPLICATIONS

This application claims the benefit of U.S. Patent Application No. 60/656,883, filed Mar. 1, 2005, the contents of which is hereby incorporated by reference in it's entirety.

### BACKGROUND

1. Field of the Invention

The present invention relates generally to formulations for therapeutic viruses and, in particular, to formulations for therapeutic viruses comprising one or more stabilizing agents, including an aqueous cosolvent, a reversible intracapsid protease inhibitor and/or a mild reducing agent.

2. Background of the Technology

Viral particles intended for human therapeutic applications must maintain their structural integrity to remain biologically active. The storage of viral vector formulations for extended periods of time, however, can result in diminished biological activity. Current viral therapeutics are typically formulated in buffers which permit their storage for extended periods of time. However, such formulations must be maintained and transported at relatively low temperatures to maintain their biological activity. Loss of activity often occurs during storage.

Storage and transport at relatively low temperature is used to minimize the loss in titer, however, this has consequences with respect to cost and the ability of viral therapeutics to be used in clinical settings that lack the facilities to store the virus under appropriate conditions.

Many known viruses, including those being employed as therapeutic viral vectors, include an external capsid (i.e. adenovirus, parvovirus, papovaviruses). To allow uncoating of external capsids during cell entry, most capsid proteins are associated non-covalently with neighboring capsid proteins. These non-covalent interactions are strong enough to maintain the assembled state of the capsid for a finite time in extra-cellular media, but are sufficiently labile under certain biological conditions (i.e. low pH, specific conformational changes due to receptor binding, enzymatic degradation, etc.). This allows for disassembly during infection. Agents that stabilize the capsid protein-protein associations find utility in therapeutic viral product formulations.

Adenovirus is known to assemble with an intracapsid viral-encoded protease. Adenovirus uncoating is a stepwise process which results in the release of viral DNA into the nucleus and dissociation of the viral capsid. Inhibitors of the cysteine protease, L3/p23, located inside the capsid have been shown to block the degradation of protein VI, indicating that the L3/p23 protease is needed to assemble virus prior to entry but also to disassemble the incoming virus. Other viruses may also rely on intracapsid proteases as part of their life cycle.

Events that trigger protease activation in formulated virus may cause degradation of the formulated virus, resulting in instability and inactivation.

Accordingly, there exists a need for viral vector formulations for therapeutic use which exhibit minimal degradation and storage stability under commercially reasonable conditions.

### SUMMARY OF THE INVENTION

One aspect of the present invention relates to an improved method for the production and storage of viruses, including but not limited to adenovirus, from cultured cells. This production method provides a novel formulation which results in improved virus stability and reduction in loss of titer during storage.

A second aspect of the present invention relates to a formulation for storing virus following processing. The formulation preserves viral activity under both frozen and non-frozen conditions, and in particular at room temperature. In one aspect of the invention, this is accomplished by inhibiting the degradation of protein VI.

In one embodiment, the formulation comprises one or more of an aqueous cosolvent, a reversible viral-encoded protease inhibitor and a mild reducing agent or other agent that prevents specific degradation of viral components.

In another embodiment, the formulation comprises an adenoviral vector, ARCA buffer and an aqueous cosolvent selected from the group consisting of propylene glycol, DMSO, PEG, sucrose, glycerol and glycofurol wherein the formulation exhibits greater stability from 2° C. to 30° C. than a formulation lacking the aqueous cosolvent.

The aqueous cosolvent may be propylene glycol at a concentration of from about 3 to 20%; glycofurol at a concentration of from about 5 to 20%; sucrose at a total concentration, ie, 10%, 20%, 30%, 40%, 50% or 60%.

In yet another embodiment, the formulation comprises an adenoviral vector which relies on a viral encoded intracapsid protease for cell entry and a reversible protease inhibitor wherein the formulation exhibits greater stability from 2° C. to 30° C. than a formulation lacking the reversible protease inhibitor.

The reversible protease inhibitor may be thioglycerol at a concentration of from about 0.5 to 2.0% or 50 to 200 mM, dimethyl sulfide at a concentration of from about 10 to 100 mM, dithiothreitol (DTF) at a concentration of from about 20-100 mM, preferably 50 mM, cysteine at a concentration of at least 1% or 150 mM, glutathione, and methionine.

The invention further provides a formulation for storage of a adenoviral vector which includes both an aqueous cosolvent and a reversible protease inhibitor (as describe above).

Preferably, the formulation exhibits greater stability when stored at 5° C. or 30° C. than a formulation lacking the addition of an aqueous cosolvent and/or a reversible protease inhibitor.

### BRIEF DESCRIPTION OF THE DRAWINGS

FIGS. 1A and 1B are graphs showing anion-exchange (AE) HPLC retention time shifts as a function of storage time for an Ad/GM-CSF (1×10¹² vp/ml), 10 wt. % PEG formulation stored at 5° C. (FIG. 1A) and for an Ad/GM-CSF (1×10¹² vp/ml), ARCA formulation stored at 25° C. (FIG. 1B).

FIG. 2A is a graph showing change in retention time (ΔRT) as a function of storage time of the AE-HPLC chromatogram peaks representing intact capsid virions (ICV) and penton-vacant virions (PVV) for an Ad/GM-CSF (1×10¹² vp/ml) formulation in ARCA buffer stored at 25° C.

FIG. 2B is a graph showing the percent of the initial intact capsid virions (ICV) and penton-vacant virions (PVV) as a function of storage time for the adenovirus formulation described in FIG. 2A.

FIG. 2C is a graph showing the percent of the initial intact capsid virions (ICV), virion particles (VP) (ICV+PVV), and GM-CSF secretion as a function of storage time for the adenovirus formulation described in FIG. 2A.

FIG. 3 is a graph of change in RT (min.) as a function of storage time for various Ad/GM-CSF (2×10¹² vp/ml) adenovirus formulations stored at 5° C. showing a formulation dependent shift in retention time.

FIGS. 4A and 4B are reversed-phase (RP) HPLC plots illustrating adenovirus protein degradation wherein FIG. 4A is a plot for a solution of Ad/GM-CSF at 1×10¹² vp/ml in ARCA at time zero and FIG. 4B is a plot for the same adenovirus solution after storage for 12 months at 15° C.

FIG. 5A is a graph showing percent intact-capsid virions (ICV₀) as a function of storage time at 30° C. for formulations of Ad/GM-CSF (1.2×10¹² vp/ml) in ARMWG (10 mM Tris, 25 mM NaCl, 2.5% glycerol, pH 8) showing the effect of various cosolvents (0.75 M) along with an ARCA control formulation.

FIG. 5B is a graph showing percent intact-capsid virions (ICV₀) as a function of storage time at 30° C. for formulations of Ad/GM-CSF (1.2×10¹² vp/ml) in ARMWG (10 mM Tris, 25 mM NaCl, 2.5% glycerol, pH 8) showing the effect of various additives (12% by weight) along with an ARCA control.

FIG. 6 is a graph showing percent intact-capsid virions (ICV₀) as a function of storage time at 5° C. for formulations of CG7060 adenovirus (2×10¹² vp/ml) in ARCA showing the effect of various concentrations of sucrose additive.

FIG. 7 is a schematic illustration of the structure of L3/p23 protease.

FIG. 8A is a graph showing percent intact-capsid virions (ICV₀) and GM-CSF secretion rate (GSR) as a function of storage time at 30° C. for solutions of Ad/GM-CSF in ARCA buffer with and without NEM pretreatment.

FIG. 8B is a graph showing percent protein VI (VI₀) as a function of storage time at 30° C. for formulations of Ad/GM-CSF in ARCA buffer with and without NEM pretreatment.

FIG. 9A is a graph showing percent intact-capsid virions (ICV₀) as a function of storage time at 30° C. for formulations of Ad/GM-CSF (1×10¹² vp/ml) in ARCA buffer showing the effect of DTT pretreatment alone or sequential to NEM pretreatment.

FIG. 9B is a graph showing percent intact-capsid virions (ICV₀) as a function of storage time at 30° C. for formulations of Ad/GM-CSF (1×10¹² vp/ml) in ARCA buffer which included 15 or 50 mM DTT.

FIG. 9C is a graph showing percent intact-capsid virions (ICV₀) as a function of storage time at 25° C. for formulations of Ad/GM-CSF (1×10¹² vp/ml) in ARCA buffer with and without 5 mM diamide (N,N, N′,N′-tetramethylazodicarboxamide) pretreatment.

FIG. 10 is a graph showing percent intact-capsid virions (ICV₀) as a function of storage time at 30° C. for formulations of Ad/GM-CSF (1×10¹² vp/ml) in ARCA buffer including various L3/p23 protease inhibitors or mild reducing agents.

FIG. 11 is a graph showing percent protein VI (VI₀) as a function of storage time at 30° C. for formulations of Ad/GM-CSF (1×10¹² vp/ml) in ARCA including selected L3/p23 protease inhibitors or mild reducing agents previously shown to results in maintenance of percent intact-capsid virions (ICV₀) over time at 30° C.

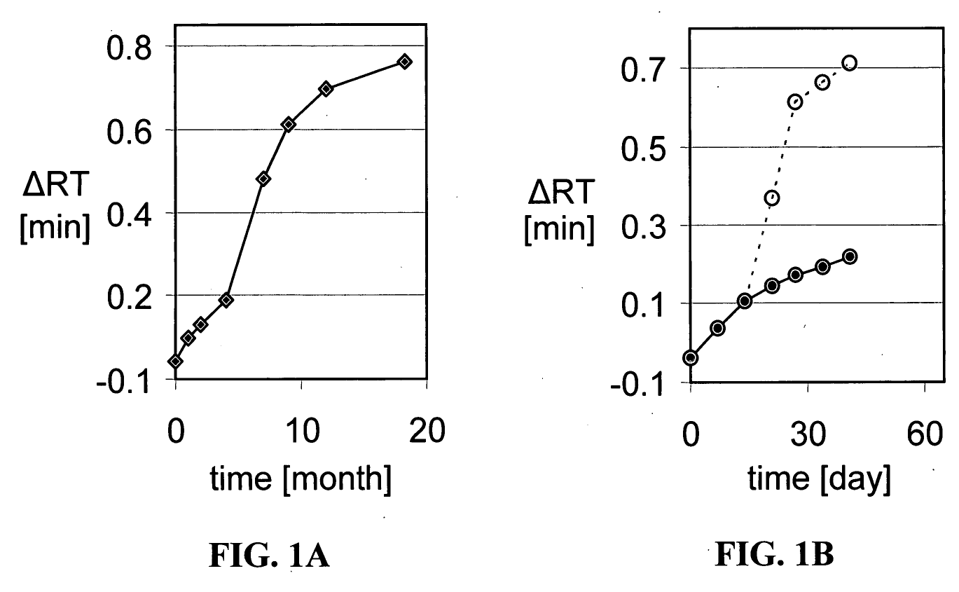

### DETAILED DESCRIPTION

Definitions

Aspects of the practice of the present invention employ, unless otherwise indicated, conventional techniques of chemistry, molecular biology, microbiology, recombinant DNA, genetics, immunology, cell biology and, cell culture, which are within the skill of those in the art.

Unless otherwise indicated, all terms used herein have the same meaning as they would to one skilled in the art and the practice of the present invention will employ, conventional techniques of chemistry, microbiology and recombinant DNA technology. All publications, patents, and patent applications cited herein are hereby incorporated by reference in their entirety for all purposes.

In describing the present invention, the following terms are employed and are intended to be defined as indicated below.

The terms “vector,” “polynucleotide vector,” “polynucleotide vector construct,” “nucleic acid vector construct,” and “vector construct” are used interchangeably herein to mean any nucleic acid construct for gene transfer, as understood by one skilled in the art.

As used herein, the term “viral vector” is used according to its art recognized meaning. It refers to a nucleic acid vector construct that includes at least one element of viral origin and may be packaged into a viral vector particle. The viral vector particles may be utilized for the purpose of transferring DNA, RNA or other nucleic acids into cells either in vitro or in vivo.

The terms “virus,” “viral particle,” “vector particle,” “viral vector particle,” and “virion” are used interchangeably and are to be understood broadly as meaning infectious viral particles that are formed when, e.g., a viral vector of the invention is transduced into an appropriate cell or cell line for the generation of infectious particles. Viral particles according to the invention may be utilized for the purpose of transferring nucleic acids into cells either in vitro or in vivo. The vectors utilized in the present invention may optionally code for a selectable marker.

As used herein, the terms “adenovirus” and “adenoviral particle” (used interchangeably) refer to any and all viruses that may be categorized as an adenovirus, including any adenovirus that infects a human or an animal, including all groups, subgroups, and serotypes.

Thus, as used herein, “adenovirus” and “adenovirus particle” refer to the virus itself or derivatives thereof and cover all serotypes and subtypes and both naturally occurring and recombinant forms, except where indicated otherwise. Such adenoviruses may be wildtype or may be modified in various ways known in the art or as disclosed herein. Such modifications include modifications to the adenovirus genome that are packaged in the particle in order to make an infectious virus. Such modifications include deletions known in the art, such as deletions in one or more of the E1a, E1b, E2a, E2b, E3, or E4 coding regions.

“Replication” and “propagation” are used interchangeably and refer to the ability of an viral vector to reproduce or proliferate. These terms are well understood in the art. For purposes of this invention, replication involves production of adenovirus proteins and is generally directed to reproduction of adenovirus. Replication can be measured using assays standard in the art and described herein, such as a virus yield assay, burst assay or plaque assay. “Replication” and “propagation” include any activity directly or indirectly involved in the process of virus manufacture, including, but not limited to, viral gene expression; production of viral proteins, nucleic acids or other components; packaging of viral components into complete viruses; and cell lysis.

The term “recombinant” as used herein with reference to nucleic acid molecules refers to a combination of nucleic acid molecules that are joined together using recombinant DNA technology into a progeny nucleic acid molecule. As used herein with reference to viruses, cells, and organisms, the terms “recombinant,” “transformed,” and “transgenic” refer to a host virus, cell, or organism into which a heterologous nucleic acid molecule has been introduced or a native nucleic acid sequence has been deleted or modified. In the case of introducing a heterologous nucleic acid molecule, the nucleic acid molecule can be stably integrated into the genome of the host or the nucleic acid molecule can also be present as an extrachromosomal molecule. Such an extrachromosomal molecule can be auto replicating. Recombinant viruses, cells, and organisms are understood to encompass not only the end product of a transformation process, but also recombinant progeny thereof. A “non transformed”, “non transgenic”, or “non recombinant” host refers to a wildtype virus, cell, or organism that does not contain the heterologous nucleic acid molecule.

The term “replication competent” as used herein means vectors and viral particles that preferentially replicate in certain types of cells or tissues but to a lesser degree or not at all in other types. In one embodiment of the invention, the vector is a replication competent adenoviral vector and/or particle that selectively replicates in tumor cells and or abnormally proliferating tissue, such as solid tumors and other neoplasms. These include the viruses disclosed in U.S. Pat. Nos. 5,677,178, 5,698,443, 5,871,726, 5,801,029, 5,998,205, and 6,432,700 and PCT publications WO 95/19434, WO 98/39465, WO 98/39467, WO 98/39466, WO 99/06576, WO 98/39464, and WO 00/15820. Such viruses may be referred to as “oncolytic viruses” or “oncolytic vectors” and may be considered to be “cytolytic” or “cytopathic” and to effect “selective cytolysis” of target cells.

The terms “replication conditional viruses”, “preferentially replicating viruses”, “specifically replicating viruses” and “selectively replicating viruses” are terms that are used interchangeably and are replication competent viral vectors and particles that preferentially replicate in certain types of cells or tissues but to a lesser degree or not at all in other types.

A “host cell” includes an individual cell or cell culture which can be or has been a recipient of a viral vector for use in practicing this invention. Host cells include progeny of a single host cell, and the progeny may not necessarily be completely identical (in morphology or in total DNA complement) to the original parent cell due to natural, accidental, or deliberate mutation and/or change. As referred to herein, a host cell includes cells transfected or infected in vivo or in vitro with a viral vector.

The term “virus permissive” means that the virus or viral vector is able to complete the entire intracellular life cycle within the cellular environment of the host cell line.

The phrases “pharmaceutically or pharmacologically acceptable” refer to molecular entities and compositions that do not produce an adverse, allergic or other untoward reaction when administered to an animal, or a human, as appropriate.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional medium or agent is incompatible with the active ingredient, its use in the therapeutic compositions is contemplated. Supplementary active ingredients also can be incorporated into the compositions.

A “stabilizing agent” as used herein refers to a component of a viral formulation that serves to enhance the stability or maintain the biological activity of the virus or viral particles in the formulation. Enhanced viral stability may be determined based on stability of percent initial intact-capsid virions (% ICV₀), percent initial protein VI (% VI₀), infectious titer assay in terms of viral particles per ml (VP/mL); hexon FACS, GM-CSF secretion level, etc.

The term “aqueous cosolvent” is used herein with reference to a water-miscible, small organic molecule such as propylene glycol, glyceryl formal, DMSO, low molecular weight polyetheylene glycol (PEG), sucrose in concentrations in excess of 5 wt. %, glycerol and glycofurol.

The term “reversible intracapsid protease inhibitor” as used herein refers to a reversible inhibitor of the encapsidated viral encoded L3/p23 cysteine protease. Examples of compounds that may act as “reversible intracapsid protease inhibitors” include thioglycerol, cysteine, glutathione, dithiothreitol (DTT), methionine and dimethyl sulfide.

The term “mild reducing agent” as used herein generally refers to thio or thiol compounds such as thiols and thioethers.

The term “ARCA” as used herein refers to a buffer for virus formulation which includes about 5% sucrose, 1% glycine, 1 mM MgCl2 and 10 mM Tris plus 0.05% polysorbate 80 (also known as “Tween 80”).

By the term “individual”, “subject”, “mammalian subject” or grammatical equivalents thereof is meant an individual mammal.

Formulations and Methods of the Invention

Various stabilizing strategies for storage of formulations of adenoviral vectors at temperatures of −20° C. to room temperature are described herein. These stabilizing strategies have been demonstrated to provide long-term physicochemical stability and maintenance of, or enhanced viral infectivity over time.

Formulations include injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain about 100 mg of human serum albumin per milliliter of phosphate buffered saline. Other pharmaceutically acceptable carriers include aqueous solutions, non-toxic excipients, including salts, preservatives, buffers and the like.

Preservatives including antimicrobial agents, anti-oxidants, chelating agents and inert gases may be included in the formulation. The pH and exact concentration of the various components in the pharmaceutical composition are adjusted according to the virus concentration, storage temperature, etc and such adjustments are known by those skilled in the art. Formulations may be further optimized for desired storage conditions according to the present invention. In one embodiment of the invention, particularly with virus formulated for clinical use, the samples are stored in liquid form, preferably at cool temperatures, usually less than about 10° C., more usually about 5° C. or lower temperatures.

For samples that are stored frozen, for example at −20° C. or −80° C., suitable buffers are as described above. Adenoviral formulations are generally stored at virus concentrations of from about 10¹¹ to about 2×10¹³ particles/ml, with greater stability typically evident upon storage in a less concentrated form.

A pharmaceutical composition of the invention is formulated to be compatible with its intended route of administration. Examples of routes of administration include parenteral, e.g., intravenous, orthotopic, intradermal, subcutaneous, intratumoral, transdermal (topical), intramuscular, intraperitoneal, transmucosal, intravenous injection, oral (e.g., inhalation) and rectal administration.

Such compositions are typically administered as pharmaceutically acceptable compositions that include physiologically acceptable carriers, buffers or other excipients. For application against tumors, direct intratumoral injection, inject of a resected tumor bed, regional (i.e., lymphatic) or general administration is contemplated. It also may be desired to perform continuous perfusion over hours or days via a catheter to a disease site, e.g., a tumor or tumor site. An effective amount is an amount sufficient to effect beneficial or desired results, including clinical results. An effective amount can be administered in one or more administrations. For purposes of this invention, an effective amount of an adenoviral vector is an amount that is sufficient to palliate, ameliorate, stabilize, reverse, slow or delay the progression of the disease state. The amount to be given is determined by the condition of the individual, the extent of disease, the route of administration, the number of doses to be administered, and the desired objective.

In one embodiment of the invention, particularly with virus formulated for clinical use, the samples are stored in liquid form, preferably at cool temperatures, usually less than about 10° C., more usually less than about 5° C. For such conditions, various stabilizing strategies for liquid formulations of adenoviral vectors are described herein. These stabilizing strategies have been demonstrated to provide long-term physicochemical stability and maintenance of, or enhanced viral infectivity over time. These strategies include the use of formulations that comprise one or more of: (1) an aqueous cosolvent; (e.g., propylene glycol or glycerol) as an agent to promote preferential hydration of capsid vertex proteins; (2) a reversible inhibitor of the encapsidated viral encoded L3/p23 protease cysteine protease (e.g., thiols or thioethers); and (3) a mild reducing agent or other agent that prevents specific degradation of viral components.

While not wishing to be bound by theory, the proposed mechanism of action of the reversible L2/p23 protease inhibitors is to prevent the digestion of protein VI (Greber, et. al. (1996); EMBO J. 15 (8)), which serves as a vertex cement protein, thus preventing disassociation of penton complexes from the vertices of the capsid. Disassociation of penton complexes from the viral capsid renders the virion biologically inactive. Therefore, preventing this disassociating event by inclusion of an aqueous cosolvent, a reversible viral-encoded protease inhibitor or a mild reducing agent in an adenoviral formulations results in stabilization of viral infectivity over time.

The mode of action of the inhibitor can be to inhibit the protease active site directly or to inhibit the active site indirectly by inducing conformational changes to the active site by interaction of the distal regions of the protease and/or the pVIc protease cofactor with the inhibitor (Jones, et. al. (1996); J. Gen. Vir. 77, 1821-1824). Evidence as to the mechanism of protease inhibition is provided in the examples and attached data. One class of L3/p23 inhibitors, mild reducing agents, also serves to inhibit oxidation of the capsid proteins, especially hexon, by scavenging oxidizing species. Anti-oxidants in general (e.g. BHT and BHA) will prevent capsid protein oxidation and are included as a component of the invention

The cosolvents may serve to promote preferential hydration (Gekko and Timasheff (1981), Biochemistry 20, 4667-4676; Arakawa and Timasheff (1982), Biochemistry 21, 6536-6544) of the surface of the vertex complex, raising the potential energy of disassociation of the penton from the peripentonal hexons, and the peripentonal hexons from the facet hexons. Cosolvents appear to provide structural stability to ribosomal complexes (Douzou (1986), Cryobiology 25, 38-47) and to assembled tubulin (Pittz and Timasheff (1978); Biochemistry 17, 615-623). Sufficiently high concentrations of cosolvents such as sucrose, glycerol, glyceryl formal propylene glycol or glycofurol may also inhibit the active site of the L3/p23 protease, and thereby serve as a member of the inhibitor class of stabilizers already described. The preferential hydration effects of cosolvents may serve to reduce the rate of conformational changes in the vertex leading to protease access to protein VI or other “unlocking” of associations between vertex proteins. Demonstration of the trigger point for the penton-disassociation event to be virion concentration dependent (as indicated by the data provided herein) suggests that an additional method of cosolvent stabilization may be due to reduced collision frequency of the virions by increasing the viscosity of the solution.

The formulations of the invention find utility in the stabilization of virus-containing crude or semi-pure process intermediates and to in situ stabilization of the virus during processing steps performed in the liquid state and leading up to the final formulated product. For examples an L3/p23 protease inhibitor may be added at the harvest step to prevent protein VI digestion during downstream processing, and moderate to high concentrations of virus-compatible cosolvents may be used during chromatography elution and tangential flow filtrations which cause high regional virus concentrations as a means to preserve virus titer.

A number of studies have been carried out to test various formulations containing one or more of an aqueous cosolvent, a reversible viral-encoded protease inhibitor and a mild reducing agent, as further described below in the Example section.

Adenoviral Vectors

The present invention contemplates the use of any and all adenoviral serotypes to construct adenoviral vectors and virus particles in the formulation according to the present invention. Adenoviral stocks that can be employed according to the invention include any adenovirus serotype. Adenovirus serotypes 1 through 51 are currently available from American Type Culture Collection (ATCC, Manassas, Va.), and the invention includes any other serotype of adenovirus available from any source. The adenoviruses that can be employed according to the invention may be of human or non human origin, such as bovine, porcine, canine, simian, avian. For instance, an adenovirus can be of subgroup A (e.g., serotypes 12, 18, 31), subgroup B (e.g., serotypes 3, 7, 11, 14, 16, 21, 34, 35, 50), subgroup C (e.g., serotypes 1, 2, 5, 6), subgroup D (e.g., serotypes 8, 9, 10, 13, 15, 17, 19, 20, 22-30, 32, 33, 36 39, 42 47, 49, 51), subgroup E (serotype 4), subgroup F (serotype 40,41), or any other adenoviral serotype. Numerous examples of human and animal adenoviruses are available in the American Type Culture Collection, found e.g., at www.atcc.org/SearchCatalogs/CellBiology.cfm.

The adenoviral vectors of the invention include replication incompetent (defective) and replication competent vectors. A replication incompetent vector does not replicate, or does so at very low levels, in the target cell, exemplified herein by the Ad/GM-CSF vector. In one aspect, a replication incompetent vector has at least one coding region in E1a, E1b, E2a, E2b or E4 inactivated, usually by deleting or mutating, part or all of the coding region. Methods for propagating these vectors are well known in the art.

In another aspect, the adenoviral vector is replication competent. Replication competent vectors are able to replicate in the target cell, exemplified herein by the CG7060 vector. Replication competent viruses include wild type viruses and viruses engineered to replicate in the target cell. These include replication specific viruses. Replication specific viruses are designed to replicate specifically or preferentially in one type of a cell as compared to another. The terms also include replication specific adenoviruses; that is, viruses that preferentially replicate in certain types of cells or tissues but to a lesser degree or not at all in other types. Such viruses are sometimes referred to as “cytolytic” or “cytopathic” viruses (or vectors), and, if they have such an effect on neoplastic cells, are referred to as “oncolytic” viruses (or vectors). In some embodiments, an adenoviral vector of the invention includes a therapeutic gene sequence, e.g., a cytokine gene sequence.

In one embodiment of the invention, the viral vector and/or particle selectively replicates in tumor cells and or abnormally proliferating tissue, such as solid tumors and other neoplasms.

In the instance of adenoviral vectors that replicate selectively in target cells, specific attenuated replication-competent viral vectors have been developed for which selective replication in cancer cells preferentially destroys those cells. Various cell-specific replication-competent adenovirus constructs, which preferentially replicate in (and thus destroy) certain cell types, are described in, for example, WO 95/19434, WO 96/17053, WO 98/39464, WO 98/39465, WO 98/39467, WO 98/39466, WO 99/06576, WO 99/25860, WO 00/15820, WO 00/46355, WO 02/067861, WO 02/06862, U.S. Patent application publication US20010053352 and U.S. Pat. Nos. 5,698,443, 5,871,726, 5,998,205, and 6,432,700. Replication-competent adenovirus vectors have been designed to selectively replicate in tumor cells.

Exemplary adenoviral vectors of the invention include, but are not limited to, DNA, DNA encapsulated in an adenovirus coat, adenoviral DNA packaged in another viral or viral like form (such as herpes simplex, and AAV), adenoviral DNA encapsulated in liposomes, adenoviral DNA complexed with polylysine, adenoviral DNA complexed with synthetic polycationic molecules, conjugated with transferrin, or complexed with compounds such as PEG, which have a variety of uses including, but not limited to, immunologically “masking” the antigenicity and/or increasing the halflife of the virus, or for conjugation to a nonviral protein.

The adenoviral vector particle may also include further modifications to the fiber protein. In one embodiment, the adenoviral vectors of the invention further comprises a targeting ligand included in a capsid protein of the particle. For examples of targeted adenoviruses, see for example, WO 00/67576, WO 99/39734, U.S. Pat. No. 6,683,170, U.S. Pat. No. 6,555,368, U.S. Pat. No. 5,922,315, U.S. Pat. No. 5,543,328, U.S. Pat. No. 5,770,442 and U.S. Pat. No. 5,846,782.

In addition, the adenoviral vectors of the present invention may also contain modifications to other viral capsid proteins. Examples of these mutations include, but are not limited to those described in U.S. Pat. Nos. 5,731,190, 6,127,525, and 5,922,315. Other modified adenoviruses are described in U.S. Pat. Nos. 6,057,155, 5,543,328 and 5,756,086.

Standard systems for generating adenoviral vectors for expression of inserted sequences are known in the art and are available from commercial sources, for example the Adeno X expression system from Clontech (Palo Alto, Calif.) (Clontechniques (January 2000) p. 10 12), the Adenovator Adenoviral Vector System and AdEasy, both from Qbiogene (Carlsbad, Calif.).

Viral Production and Purification

Host cells for use in generating a viral preparation of the invention are capable of supporting replication of the candidate virus. As defined herein, “host cell” includes an individual cell or cell culture which can be or has been infected by a virus. Host cells include progeny of a single host cell, and the progeny may not necessarily be completely identical (in morphology or in total DNA complement) to the original parental cell due to natural, accidental, or deliberate mutation and/or change. A host cell includes cells transfected or infected in vivo or in vitro with a viral vector of this invention.

Host cells according to the present invention are derived from a mammalian cell and, preferably, from a primate cell. Although various primate cells are preferred and human cells are most preferred, any type of cell that is capable of supporting replication of the virus is acceptable in the practice of the invention. Cell types for use in practicing the invention, include, but are not limited to, Vero cells, CHO cells or any eukaryotic cells which are permissive for the type of virus being produced. A candidate cell line may be tested for its ability to support virus replication by methods known in the art, e.g. by contacting a layer of uninfected cells, or cells infected with one or more helper viruses, with virus particles, followed by incubation of the cells and determination of viral replication.

A variety of host cells are capable of supporting replication of adenovirus. Although various primate cells are preferred and human cells are most preferred, any type of cell that is capable of supporting replication of the virus is acceptable in the practice of the invention. A preferred cell line for commercial scale production of adenovirus is the HeLa-S3 cell line as described for example in U.S. application Ser. No. 10/824,796, expressly incorporated by reference herein.

Additional preferred cell lines for commercial scale production of adenovirus are A549 cells, PERC6 cells and human 293 embryonic kidney cells, which expresses the adenoviral EIA and EIB gene products, and the like. Cell lines capable of producing appropriately targeted adenovirus include human LNCaP (prostate carcinoma), HBL-100 (breast epithelia), OVCAR-3 (ovarian carcinoma), and the like.

Cell Culture

The host cells are usually grown in perfused systems, which allow for the maintenance of a good culture environment of pH, CO2 and O2 while the cells are growing. Perfusion allows active metabolites to be removed, while the nutrients are being supplied. The appropriate medium and conditions suitable for culture of cell lines useful in the production of viral vectors according to the present invention are well known in the art, and any suitable medium can be utilized, e.g. RPMI, DMEM, etc. The medium may contain serum, e.g. fetal bovine serum or may be serum-free. Serum weaning adaptation of anchorage-dependent cells into serum-free suspension culture has been used for the production of recombinant proteins and viral vaccines, and may find use in the production of viral vectors.

In certain embodiments, it may be useful to employ selection systems that preclude growth of undesired cells. This may be accomplished by virtue of permanently transforming a cell line with a selectable marker-encoding vector or by transducing or infecting a cell line with a viral vector that encodes a selectable marker. In either situation, culture of the transformed/transduced cell with an appropriate drug or selective compound will result in the selective replication of those cells carrying the marker. Selective replication of cells carrying the marker means that culture of transformed/transduced cells in the presence of an appropriate type and concentration of drug or selective compound results in either preferential or exclusive replication of cells that carry the marker relative to cells that do not carry the marker. Examples of markers include, but are not limited to, HSV thymidine kinase (TK), hypoxanthine-guanine phosphoribosyltransferase (HGPRT) and adenine phosphoribosyltransferase (APRT) genes, in TK, HGPRT- or APRT-cells, respectively. Also, anti-metabolite resistance can be used as the basis of selection for dihydrofolate reductase (DHFR), that confers resistance to methotrexate; gpt, that confers resistance to mycophenolic acid; neo, that confers resistance to the aminoglycoside G418; and the hph gene, that confers resistance to hygromycin.

Serum weaning adaptation of anchorage-dependent cells into serum-free suspension cultures has been used for the production of recombinant proteins (Berg et al., BioTechniques, 14:972-978, 1993) and viral vaccines (Perrin et al., Vaccine, 13:1244-1250, 1995; Gilbert) using 293 cells (Williamsburg BioProcessing Conference, Nov. 18-21, 1996; WO98/22588) and A549 cells (Morris et al., Williamsburg BioProcessing Conference, Nov. 18-21, 1996).

Some anchorage-dependent cells may be adapted to suspension culture, which can facilitate commercial scale production. The present invention may rely on use of bioreactor technology for production of virus. Growing cells in a bioreactor allows for commercial scale production of cells capable of being infected by a viral vector for use in the methods and formulations of the present invention. By operating the system under perfusion conditions and applying an improved scheme for purification, the invention provides a strategy that is easily scaleable to produce sufficient quantities of commercially useful viral vectors.

Bioreactors have been widely used for the production of biological products from both suspension and anchorage dependent animal cell cultures. Perfusion of fresh medium through the culture can be achieved by retaining the cells with a variety of devices, e.g. fiber disks, fine mesh spin filter, hollow fiber or flat plate membrane filters, settling tubes, etc. A simple perfusion process has an inflow of medium and an outflow of cells and products. Culture medium is fed to the reactor at a predetermined and constant rate which maintains the dilution rate of the culture at a value less than the maximum specific growth rate of the cells.

In the production of a virus, host cells are infected with the virus by contacting the cells with the virus under physiological conditions permitting the uptake of virus. Cells may be infected at a high multiplicity of infection (MOI) in order to optimize yield. The host cell replicates the virus, which can be harvested at 2-5 days post infection.

Cell Lysate

A critical step in the process of viral production is release of the virus from the host cells upon lysis of the cell membrane. Traditional methods for lysing cells, such as mechanical agitation (e.g., high pressure extrusion, solid shear, liquid shear, or sonication) and freeze-thaw cycles, may damage the virus and often result in a lower yield of biologically active viral product. Another lysis method is detergent lysis, which typically relies upon the addition of non-ionic detergent to the infected cells, at a final concentration of about 0.5%-2.5 weight/volume. Commonly used non-anionic detergents include the Triton™ family of detergents (e.g. Triton™ X-15; Triton™ X-35; Triton™ X-45; Triton™ X-100; Triton™ X-102; Triton™ X-114; Triton™ X-165, all of these heterogeneous detergents have a branched 8-carbon chain attached to an aromatic ring), the Tween™ detergents which are a family of nondenaturing, nonionic polyoxyethylene sorbitan esters of fatty acids, and the zwitterionic detergent CHAPS, which is a sulfobetaine derivative of cholic acid. Such non-ionic detergents, however, are also potentially damaging to the virus.

Virus infected cells may be harvested and lysed using a lysis reagent containing one or more non-ionic surfactants that are known to associate with cell-membrane proteins and help their solubilization (see, e.g., Taylor et al., Biochim Biophys Acta. 1612:65-75, 2003; Santoni et al., Proteomics, 3:249-253, 2003; and Hazard et al., Arch Biochem Biophys. 407:117-124, 2002). Such non-ionic surfactants may result in less damage to the virus during purification and increase stability during storage. The non-ionic surfactant in the lysis reagent preferably has both hydrophilic and lipophilic sections. Examples of the non-ionic surfactants include, but are not limited to, alkyl substituted mono-, di-, and polysaccharides, cyclic alkyl substituted mono-, di-, and polysaccharides alkyl alcohol, polyoxyethylene ethers, dialkyl-glycerols, and isomers thereof.

Any mono-, di-, or poly-saccharide having a lipophilic substituent can be used as the non-ionic surfactant in the lysis reagent of the invention. Exemplary di-saccharide compounds include sucrose, lactose, maltose, isomaltose, trehalose, and cellobiose. The lipophilic substituent preferably comprises an alkyl or alkenyl group. According to a preferred embodiment of the invention, the lipophilic substituent is an alkanoic acid residue.

The lipophilic substituent can be linear (e.g., a straight chain n-alkane or alkene) or non-linear (e.g., cyclic or branched chain alkanes or alkenes). The lipophilic substituent can also be an alkanoic acid residue. The length of the lipophilic substituent can be varied to achieve the desired hydrophilic-lipophilic balance.

Preferred non-ionic surfactant include alkyl substituted monosaccharides, alkyl substituted disaccharides, alkyl substituted polysaccharides, cyclic alkyl substituted monosaccharides, cyclic alkyl substituted disaccharides, cyclic alkyl substituted polysaccharides alkyl alcohol, polyoxyethylene ethers, dialkyl-glycerols, and isomers thereof, such as n-Dodecyl-b-D-maltoside, n-Dodecyl-b-D-maltoside, n-Dodecyl-b-L-maltoside, n-Dodecyl-b-L-maltoside, 6-cyclohexylhexyl-b-D-maltoside, 6-cyclohexylhexyl-b-D-maltoside, 6-cyclohexylhexyl-b-L-maltoside, 6-cyclohexylhexyl-b-L-maltoside, 6-cyclohexylhexyl-b-D-maltoside, sucrose monolaurate, n-tridecyl-b-D-maltoside, n-tridecyl-b-D-maltoside, n-tridecyl-b-L-maltoside, n-tridecyl-b-L-maltoside, n-tetradecyl-b-D-maltoside, n-tetradecyl-b-D-maltoside, n-tetradecyl-b-L-maltoside, n-tetradecyl-b-L-maltoside and polidocanol, as further described in U.S. patent application Ser. Nos. 10/743,813 and 60/631,434, each expressly incorporated by reference herein.

The lysis agent is contacted with the cells for a period of time sufficient to lyse the cells and remove additional adherent cells from the system. In such systems, before purification of the virus, the crude viral lysate is generally clarified i.e., the membrane fragments are removed. Clarification is achieved by the use of depth filters consisting of a packed column of a non-absorbent material of certain porosity such that the bigger membrane debris is retained without the loss of viral particles. Depth filters are selected on the basis of mechanical retention of particles, absorption characteristics, pH value, surface quality, thickness and strength of the filter. Commercially available cartridges combine several types of filters, e.g. polypropylene, glass fibers, nitrocellulose, and the like.

Virus Purification

Techniques used for isolating infectious virus from cell lysates are well-known in the art. For example, the virus can be isolated by either density gradient centrifugation or chromatography (see e.g., U.S. Pat. Nos. 6,194,191 and 6,689,600). The appropriate density condition for centrifugation is utilized depending on the kind of the viral vector to be separated and information generally known in the art.

In order to make purification of viruses a scalable process, it is preferable to use procedures such as chromatography, which remove cellular debris from the cell lysate without centrifugation. In standard processes, viral particles are separated from clarified cell lysates by ion exchange chromatography on a filter cartridge, numerous examples of which are known in the art. A variety of commercially available chromatographic materials can be used for viral purification. Useful support matrices include, but are not limited to, polymeric substances such as cellulose or silica gel type resins or membranes or cross-linked polysaccharides (e.g. agarose) or other resins. The chromatographic materials can further comprise various functional or active groups attached to the matrices that are useful in separating biological molecules.

As such, virus may be purified by use of affinity groups bound to support matrices with which the virus interacts via various non-covalent mechanisms, followed by subsequent removal. Preferred separation methods include ion-exchange (in particular, anion-exchange). Other specific affinity groups include heparin and virus-specific antibodies bound to a support matrix. Of consideration in the choice of affinity groups in virus purification is the avidity with which the virus interacts with the chosen affinity group and ease of removal therefrom without damaging the biological function/infectivity of the viral particles. Exemplary polymeric materials include the products Heparin Sepharose High Performance (Pharmacia); macroporous hydroxyapatite such as Macro-Prep Ceramic Hydroxyapatite (Bio-Rad, Richmond, Calif.); and cellufine sulfate (Amicon). Affinity ligands which may be used to purify virus include anti-virus antibodies attached to suitable resins as others known to those skilled in the art.

Anion-exchange chromatography may be performed utilizing various functional moieties known in the art including, but not limited to, DEAE, (diethyl aminoethyl), QAE (quaternary aminoethyl), and Q (quaternary ammonium). These functional moieties may be attached to any suitable resin including cellulose and silica resins. For example, DEAE may be attached to various resins, including cellulose resins, in columns such as DEAE-MemSep™ (Millipore, Bedford, Mass.). Sartobind™ membrane absorbers (Sartorius, Edgewood, N.J.) and silica resins such as ACTI-MOD™ (American International Chemical, Natick, Mass.). Exemplary resins also include polystyrene cross-linked with divinylbenzene beads, as found in Pharmacia Source Q, and dextran attached to highly cross-linked spherical agarose beads, as found in Pharmacia Q-Sepharose XL. (See, e.g., WO 00/40702, expressly incorporated by reference herein).

Cation-exchange chromatography also may be used for virus purification, including, but not limited to, the use of such columns as SP MemSep™ (Millipore, Bedford, Mass.), CM MemSep™ (Millipore, Bedford, Mass.), Fractogel™ SO3 (EM Separation Technology, Gibbstown, N.J.) and Macroprep S™ (BioRad, Melville, N.Y.), as well as heparin-based resins. Heparin ACTI-MOD™ Cartridge (American International Chemical Inc., Natick, Mass.), and POROS™ Perfusion chromatography media (Boehringer Mannheim) represent additional examples.

Nuclease Treatment

The eluted virus may be treated with a nuclease. Treatment with a nuclease after chromatography, as compared to immediately post-harvest, minimizes the amount of nuclease required. A second chromatography step after nuclease treatment may be included to remove fragmented DNA and the nuclease.

Many nucleases are known in the art, with preferred nucleases including one or a combination of broad specificity endonucleases, e.g. enzyme classification 3.1.27.5 (pancreatic ribonuclease) and 3.1.31.1 (micrococcal nuclease); and the like. Benzonase™, a genetically engineered enzyme with both DNase and RNase activity is particularly useful in this step of the viral purification process. The ability of Benzonase™ to rapidly hydrolyze nucleic acids makes the enzyme useful for reducing cell lysate viscosity, and for reducing the nucleic acid load during purification, thus eliminating interference and improving yield. Upon complete digestion, free nucleic acids present in solution are reduced to oligonucleotides 2 to 4 bases in length. Following nuclease digestion, the virus may be run for a second time on an ion exchange filter, where the filter may be the same or different as the first filter.

Filtration/Sterilization

The eluted virus is optionally concentrated and diafiltered by conventional methods, e.g. with a hollow fiber concentrator. In a final preparation for use, the purified virus sample may be sterile filtered, e.g. for clinical use. A variety of filters suitable for this purpose are known in the art, e.g. nitrocellulose membrane filters; cellulose acetate membrane filters; PVDF (modified polyvinylidene fluoride) membrane filters; and the like. Preferred are PVDF membrane filters (for example Millipore Millipak filters). The yield may be improved by pre-washing the filters using a buffer, e.g. a pharmaceutically acceptable excipient. It has been found that yield is reduced by binding of virus to the filter, where the binding is saturated after a certain level. Therefore, yield can be improved by loading a higher number of particles, so that the percentage loss is minimized. The particular conditions for sterilization by filtration are appropriately set depending on the degree of purification, concentration of the viral vector to be used, and the like.

Characterization of Virus

Methods for determining the potency and purity of enriched or isolated viruses are well known in the art. For example, quantitative characterization of infectivity of a virus can be determined by measuring the expression of a virus-encoded protein in a permissive cell. Typically, permissive cells are infected with the virus in a serial dilution, incubated for a certain period of time (e.g., 1-2 days), and then screened for expression of the virus-coded gene, which can be a viral gene (e.g. the DNA binding protein of adenovirus) or a transgene carried by the virus (e.g., beta-galactosidase). The infectivity of certain viruses, such as adenovirus and vaccinia virus, can also be determined by plaque assay.

The purity of the virus can be determined by reverse-phase high-performance liquid chromatography (RP-HPLC). During chromatography, intact virus dissociates into its structural components, i.e., hexon, penton base (Pb), and fiber, yielding a characteristic fingerprint that may be shifted based on integrity of the various components. The viral concentration can be also measured through quantification of structural proteins (see e.g., Lehmberg et al., J Chromatogr B Biomed Sci Appl. 732:411-423, 1999; and Roitsch et al., J Chromatogr B Biomed Sci Appl., 752:263-280, 2001). See Table 1.

Anion exchange chromatography (AEX) may also be used to determine the purity, concentration and potency of viruses. This method has been used to quantify adenovirus particles in either crude lysates or highly pure samples (Shabram et al., Hum Gen Ther., 8:453-465, 1997). It can be used to assess particles in both dilute and concentrated samples over a wide dynamic range.

### EXAMPLES

Stability of Various Adenovirus Formulations

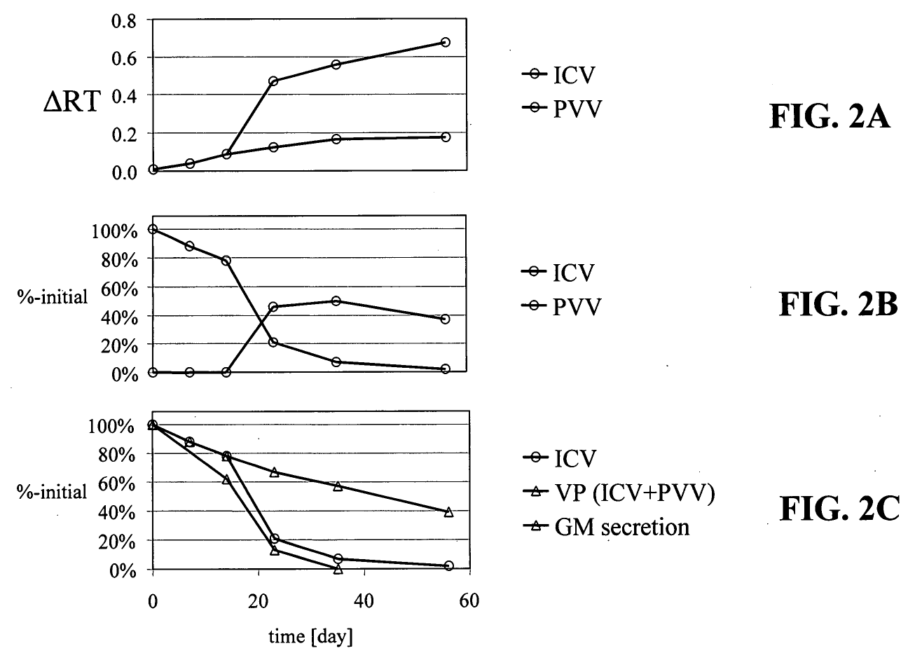

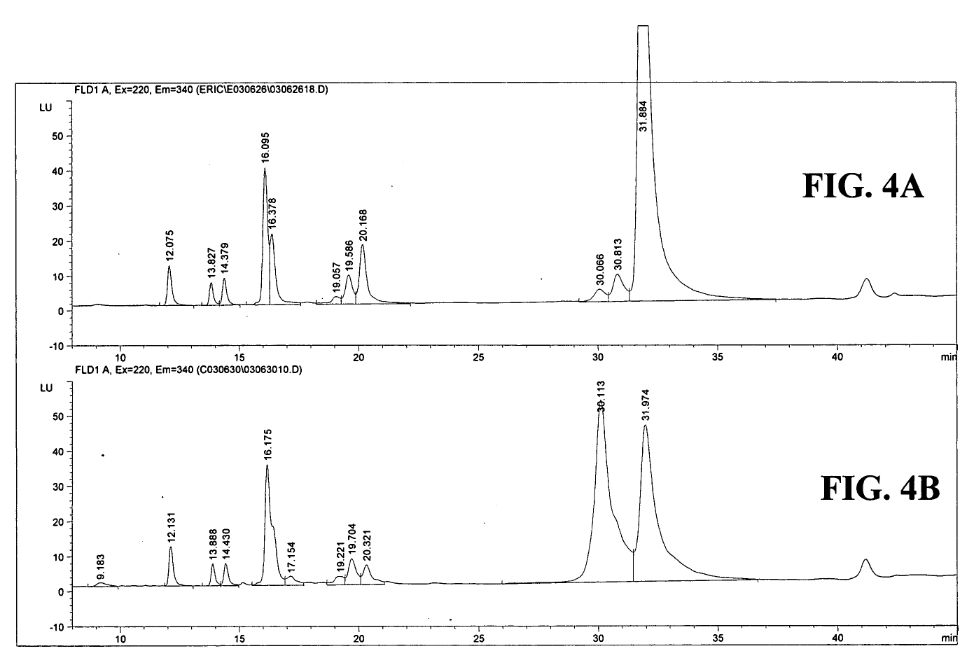

Cultured HEK 293 cells were infected with adenovirus of a chosen serotype and harvested by centrifugation. The cell pellet was resuspended in lysis buffer and formulated with various buffers described below. ARCA buffer includes 5% sucrose, 1% glycine, 1 mM MgCl2 and 10 mM Tris, plus 0.05% polysorbate 80 (also known as “Tween 80”). Each formulation was sterile-filtered through a 0.2 micron filter and filled in 1-ml glass vials with Teflon coated, silicon rubber stoppers. The vials were stored at 5° C., 25° C., or 30° C. Storage at 25° C., or 30° C. is considered to be storage at room temperature. At selected time points, samples of each formulation were studied by anion exchange chromatography, reversed phase chromatography, etc. The AE-HPLC results confirm that more changes in peak patterns had occurred in samples kept in ARCA buffer alone than in ARCA buffer components plus an aqueous cosolvent alone or in combination with a mild reducing agent or a reversible protease inhibitor. See, for example FIGS. 2A-C and FIGS. 4A and B.

AE-HPLC was used to evaluate the percent of intact components, hexon, penton base (Pb) and fiber over time indicates that the percent intact penton base and fiber is significantly lower at longer storage times, whereas the percent intact hexon remains relatively stable (e.g., Table 1).

For example AE-HPLC chromatograms for a solution of Ad/GM-CSF (1×10¹² vp/ml) in GTS/ARMWG buffer stored at 37° C. at time zero demonstrate a peak at 6.113 minutes for only ICV. At an intermediate storage time two peaks at 6.106 and 6.650 minutes are present (one for IVC and one for PVV) and at a later storage time the peak at 6.701 minutes for PVV predominates.

Table 1 shows the transition between live virus (as indicated by the peak at approximately 6.1 minutes) to non-infectious virus (as indicated by the peak at approximately 6.7 minutes) over time when a preparation of Ad/GM-CSF (1×10¹² vp/ml) is stored in GTS/ARMWG buffer stored at 37° C. Table 1 also shows the composition of HPLC peaks having a shift in retention time of 0, 0.4 and 0.7 minutes indication a transition between live virus (as indicated by hexon, penton base (Pb) and intact fiber content). The peak with no shift in retention time correlates with a high percentage of live virus and % intact capsid virions (ICV), while the peak with a shift in retention time of 0.7 minutes correlates with non-infectious virus (as indicated by penton-vacant virions or PVV).

TABLE 1
AE-HPLC evaluation of percent hexon (RP),
penton base (Pb) and in tact fiber (WB)

| RT Shift | % intact | % intact | % intact fiber |
|---|---|---|---|
| (minutes) | hexon (RP) | Pb (RP) | (WB) |
| 0 | 94 | 100 | 87 |
| 0.4 | 95 | 100 | 82 |
| 0.7 | 85 | 32 | 26 |

Consistent with Table 1, FIG. 2A shows that a formulation of 1×10¹² vp/ml of Ad/GM-CSF stored in ARCA buffer at 25° C. degrades over time such that the % intact capsid virions (ICV) decreases consistent with an increase in penton-vacant virions (PVV) and GM-CSF secretion.

FIG. 2C indicates that the drop-off in GM-CSF secretion correlates with decrease in the percentage of intact capsid virions (ICV) indicating that the percentage of ICV is indicative of the capability of an Ad/GM-CSF viral preparation of to effect secretion of GM-CSF.

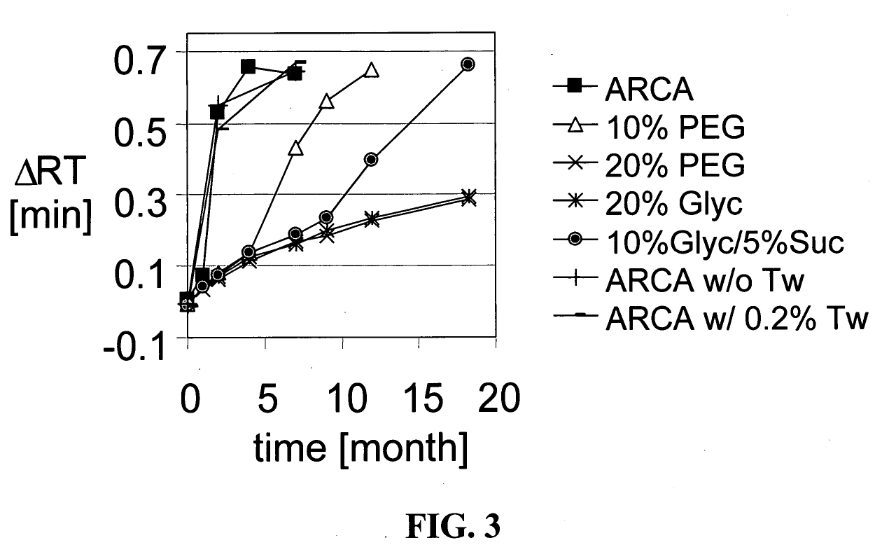

A number of studies were carried out where aqueous co-solvents were evaluated for their effect on stability of adenoviral formulations. As shown in FIG. 3, formulations including 20% PEG, 20% glycerol led to a only small change in retention time, indicating enhanced viral stability.

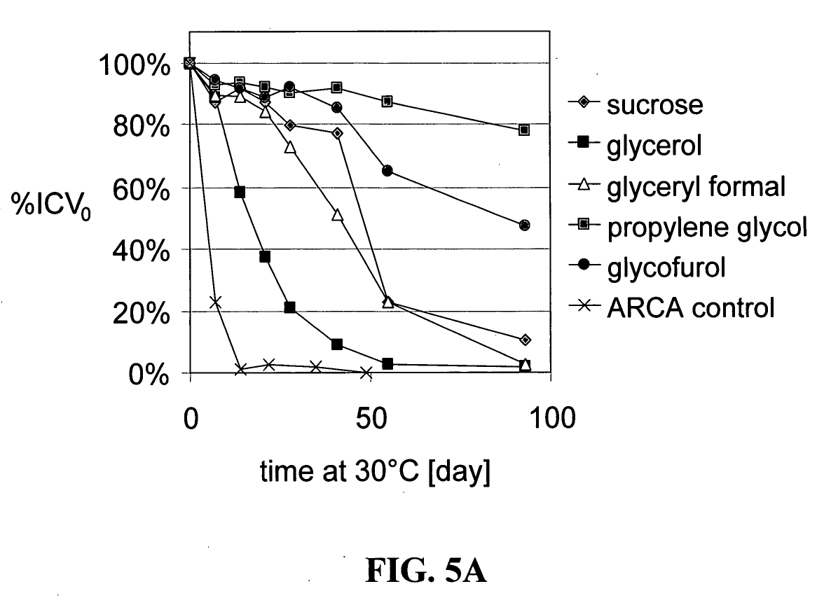

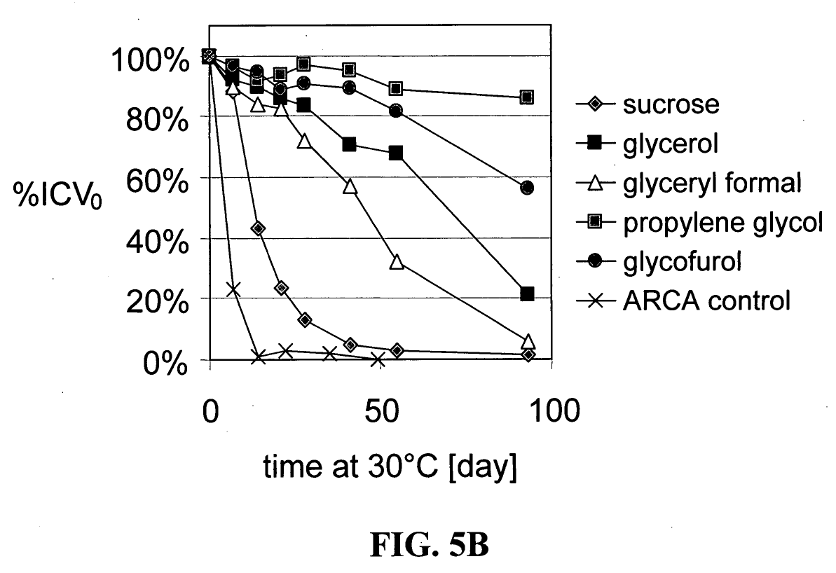

Further studies on the percent of intact-capsid virions (% ICV₀) as a function of storage time at 30° C. for solutions of Ad/GM-CSF (1.2×10¹² vp/ml) in ARMWG formulation (10 mM Tris, 25 mM NaCl, 2.5% glycerol, pH 8) were carried out to evaluate the effect of various stabilizing additives at concentrations of 0.75 M (FIG. 5A) or 12% by weight (FIG. 5B) as compared to an ARCA control. As can be seen from FIG. 5A and FIG. 5B, formulations including propylene glycol or glycofurol exhibited the greatest stability and the ARCA control formulation exhibited the lowest storage stability following at least 50 days at 30° C.

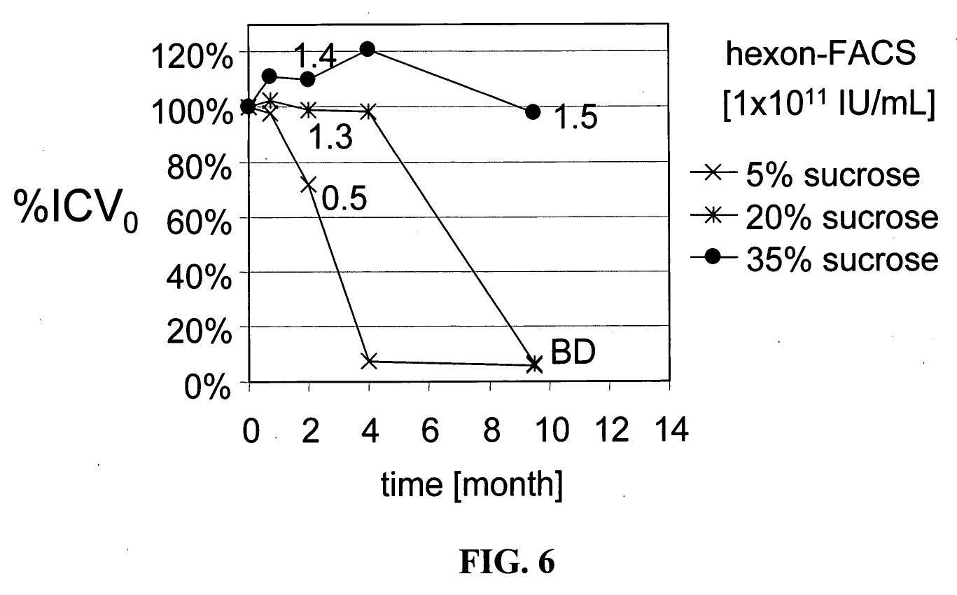

The effect of various concentrations of sucrose on virus stability was also evaluated based on the percent of intact-capsid virions (% ICV₀) as a function of storage time at 30° C. for solutions of Ad/GM-CSF (1.2×10¹² vp/ml). The 5 wt. % sucrose formulation is a standard ARCA formulation (due to the fact that ARCA contains 5 wt. % sucrose). As can be seen from FIG. 6, higher sucrose concentrations resulted in greater adenovirus stability.

FIGS. 5A, 5B and 6 indicate that the drop-off in % ICV seen when virus is stored in ARCA alone can be decreased by inclusion of various aqueous cosolvents such as propylene glycol and glycofurol.

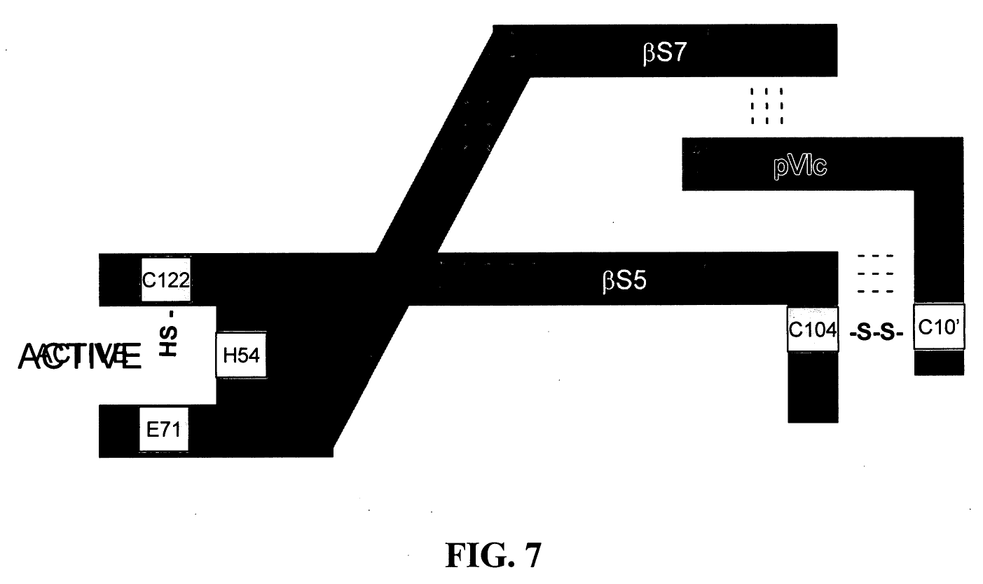

A number of studies were carried out in an attempt to understand the mechanism by which reversible protease inhibitors and/or a mild reducing agent contribute to the stability of adenoviral formulations. In order to illustrate the potential mechanism, viral particles were pretreated prior to addition to a particular formulation. A schematic illustration of the structure of the L3/p23 protease is provided in FIG. 7, which indicates the presence of a disulfide bond and a free thiol. Pretreatment studies are described herein merely for purposes of exemplifying the potential mechanism of action of particular types of agents and are not intended to be included in a formulation the invention. For instance, NEM or N-ethylmaleimide is a non-reversible protease inhibitor and diamide is an oxidizing agent. Both were employed for proof of concept studies and are not intended to be a component of a compositions for therapeutic use. DTT is a reducing agent that reversibly inhibits disulfide bonds to maintain free thiols and constitutes an aspect of the present invention.

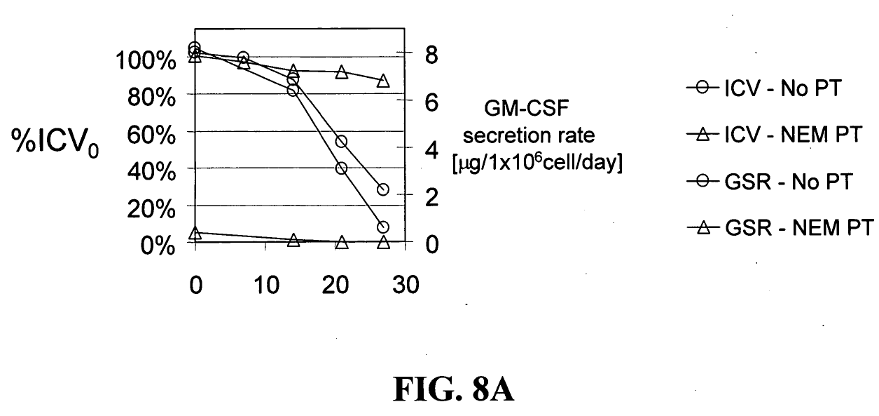

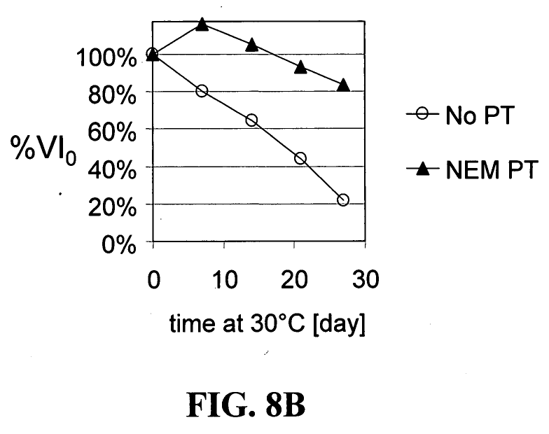

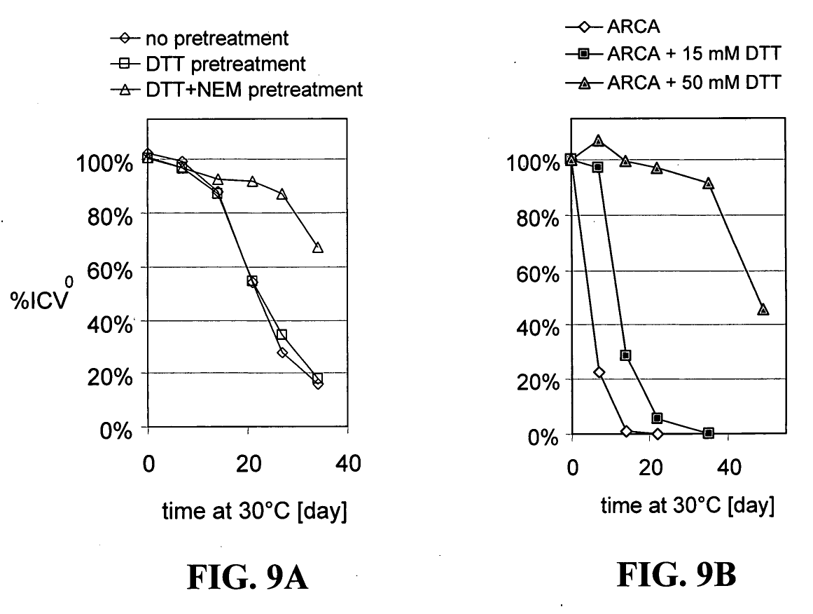

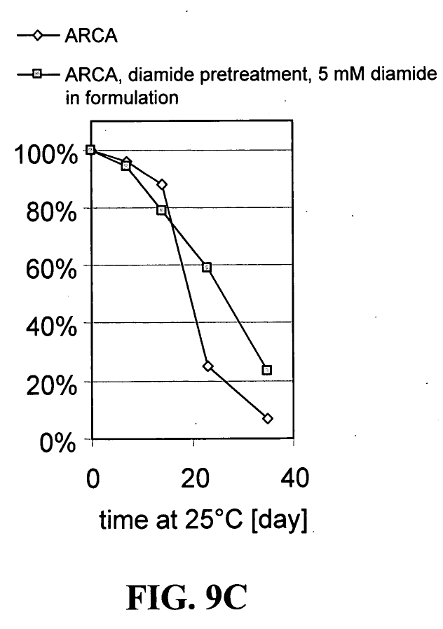

FIGS. 8A, 8B and 9A-9C illustrate that pretreatment with NEM, DTT and diamide, minimizes the decrease in % ICV seen over time when virus is stored in ARCA buffer at 30° C., suggesting that inhibition of viral proteases may be a means to achieve enhanced viral stability when viral preparations are stored in ARCA buffer. The effect of no pretreatment was compared to DTT pretreatment at 15 and 50 mM DTT for formulations of Ad/GM-CSF (1×10¹² vp/ml) in ARCA. The percent intact-capsid virions (ICV₀) as a function of storage time at 30° C. indicated that the formulation with 50 mM DTT exhibited greater stability than the formulation with no DTT and the formulation and with 15 mM DTT. (FIG. 9B).

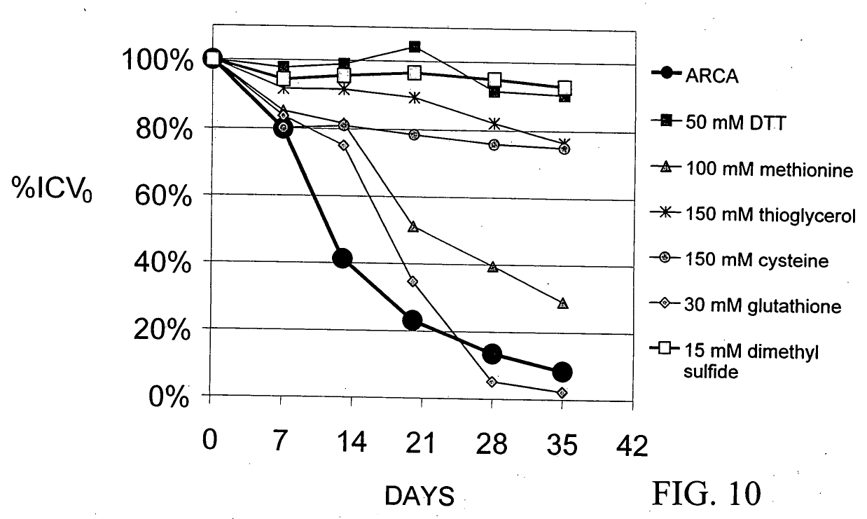

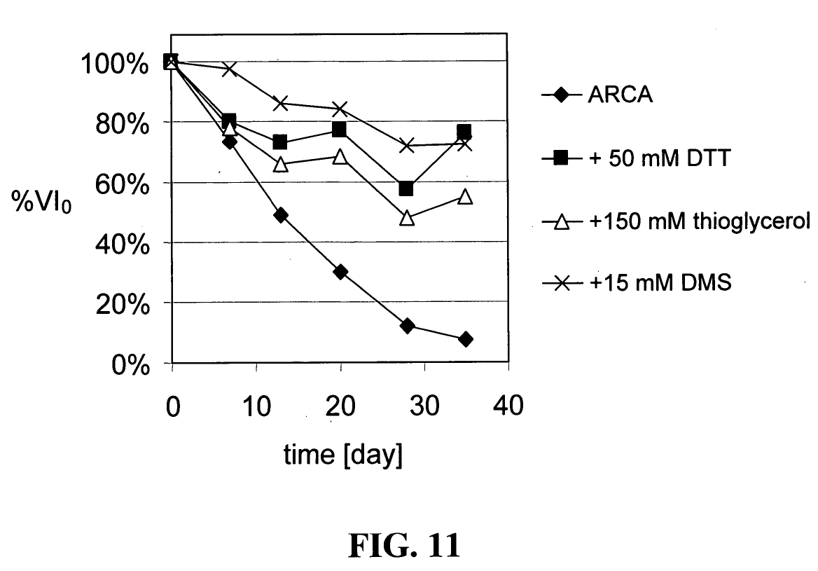

FIGS. 10 and 11 show the percent initial intact-capsid virions (% ICV₀) and percent initial protein VI (% VI0), respectively, as a function of storage time at 30° C. for formulations of Ad/GM-CSF (1×10¹² vp/ml) in ARCA formulation containing various protease inhibitors. The results show that formulations containing 50 mM DTT, 150 mM cysteine and 15 mM dimethyl sulfide exhibited stability based on the results of analyses for both percent initial intact-capsid virions (% ICV₀) and percent initial protein VI (% VI₀).

Table 2 indicates that formulations including 50 mM DTT, 150 mM thioglycerol and 15 mM dimethyl sulfide, respectively, also exhibited stability based on GM-CSF secretion as a function of storage time at 30° C. as compared to an ARCA formulation which lacks a protease inhibitor.

TABLE 2
Formulation Stability

| Formulation | % ICV₀ | % GSR0 |
|---|---|---|
| ARCA | 41% | 31% |
| +50 mM DTT | 99% | 88% |
| +150 mM thioglycerol | 92% | 83% |
| +15 mM DMS | 96% | 89% |

A number of different formulations (listed in Table 3) were shown to enhance the stability of a stock of Ad/GM-CSF virus as determined by percent initial intact-capsid virions (% ICV0) as a function of storage time at 30° C.; percent initial protein VI (% VI0) as a function of storage time at 30° C.; and percent of initial GM-CSF secretion rate (GSR). The stability for each formulation listed in Table 3 are provided in Table 4.

TABLE 3
Formulation Abbreviations.

| # | Formulation |
|---|---|
| 1 | ARCA (5% Sucrose, 1% Glycine, 1 mM MgCl₂, |
| | 10 mM Tris plus 0.05% polysorbate 80) |
| 2 | ARCA + 30% more sucrose + 1% thioglycerol |
| 3 | ARCA + 15% more sucrose + 1% thioglycerol |
| 4 | 10% glycerol in ARCA − sucrose/polysorbate + 1% thioglycerol |
| 5 | 10% glycerol in ARCA − sucrose/polysorbate + 15 mM DMS |
| 6 | 5% glycerol in ARCA − sucrose/polysorbate + 1% thioglycerol |
| 7 | 10% propylene glycol in ARCA − sucrose/polysorbate + |
| | 1% thioglycerol |
| 8 | 5% propylene glycol in ARCA − sucrose/polysorbate + |
| | 1% thioglycerol |
| 9 | 5% propylene glycol in ARCA − sucrose/polysorbate + |
| | 15 mM DMS |
| 10 | 5% propylene glycol in ARCA − sucrose/polysorbate + |
| | 15 mM DMS + 1% thioglycerol |

TABLE 4
Formulation Stability

| | % ICV₀ | | | % VI₀ | | | % GSR₀ |
|---|---|---|---|---|---|---|---|
| # | 16 days | 30 days | 43 days | 16 days | 30 days | 43 days | 43 days |
| 1 | 26% | 1% | 1% | 44% | 0% | 0% | 0% |
| 2 | 91% | 96% | 89% | 70% | 74% | 67% | 51% |
| 3 | 92% | 95% | 79% | 83% | 75% | 51% | 24% |
| 4 | 94% | 97% | 92% | 81% | 76% | 67% | 39% |
| 5 | 90% | 98% | 88% | 83% | 70% | 55% | 29% |
| 6 | 89% | 96% | 70% | 75% | 72% | 38% | 20% |
| 7 | 96% | 101% | 98% | 79% | 75% | 56% | 56% |
| 8 | 94% | 99% | 95% | 70% | 67% | 49% | 59% |
| 9 | 93% | 97% | 95% | 69% | 60% | 55% | 40% |
| 10 | 94% | 97% | 95% | 68% | 67% | 52% | 50% |

Table 5 illustrates the long-term stability of an Ad/GM-CSF (CG6444) formulation (50 wt. % glycerol, 10 mM Tris, pH of 7.4) at −20° C. even after extended periods of storage (approximately 36.5 months).

TABLE 5
Long-Term Stability of CG6444 formulated in 50% Glycerol,
10 mM Tris, pH 7.4 and stored at −20° C.

| | | | | 0.2 mm | | | |
|---|---|---|---|---|---|---|---|
| | AEX-HPLC | | | Filtration | GM-CSF | Hexon-FACS | |
| Time | Titer | Percent | D RT | Recovery | Secretion | Inf. Titer | Inf. Ratio |
| (month) | (VP/mL) | Initial | (min) | (%) | (SU100) | (IU/mL) | (VP/IU) |
| 0 | 1.1E+12 | 100% | 0.035 | 96% | 2.7 | 5.3E+09 | 189 |
| 0.5 | 1.1E+12 | 100% | 0.021 | 95% | 21.2 | 4.3E+10 | 23 |
| 1 | 1.1E+12 | 97% | 0.050 | 98% | 17.3 | 4.3E+10 | 23 |
| 3 | 1.2E+12 | 103% | 0.062 | 104% | 9.7 | 2.5E+10 | 40 |
| 6 | 9.5E+11 | 84% | 0.091 | 101% | 7.0 | 1.9E+10 | 53 |
| 9 | 9.3E+11 | 82% | 0.054 | 95% | 6.9 | 2.8E+10 | 36 |
| 15 | 1.1E+12 | 93% | 0.035 | 93% | 11.5 | 4.3E+10 | 23 |
| 27.3 | 9.3E+11 | 82% | −0.005 | ND | 11.6 | 2.18E+10 | 46 |
| 36.5 | 1.0E+12 | 92% | 0.029 | ND | ND | ND | ND |

A number of additional studies were carried out where aqueous co-solvents were evaluated for their effect on stability of adenoviral formulations.

A number of different cosolvent formulations (listed in Table 6) were used to test CG0070 viral stocks containing 1E12 VP/mL and each was shown to enhance the stability of a stock of CG0070 virus as a function of storage time at −20° C. for up to 16 months. The results of titer analysis and stability studies for each formulation listed in Table 6 are provided in Table 7. Each formulation tested showed good stability for up to 16 months.

TABLE 6
Formulations Tested.

| | Titer | | Tris | | | Gly- |
|---|---|---|---|---|---|---|
| No. | (VP/mL) | pH | Buffer | MgCl2 | Cosolvent | cine |
| 1 | 1.0E+12 | 7.8 | 6 mM | 0.6 mM | 50% (v/v) glycerol | 0.6% |
| | | | | | | (w/v) |
| 2 | 1.0E+12 | 7.8 | 6 mM | 0.6 mM | 50% (v/v) propylene | 0.6% |
| | | | | | glycol | (w/v) |
| 3 | 1.0E+12 | 7.8 | 6 mM | 0.6 mM | 50% (v/v) glycerol | — |
| 4 | 1.0E+12 | 7.8 | 6 mM | 0.6 mM | 50% (v/v) propylene | — |
| | | | | | glycol | |
| 5 | 1.0E+12 | 7.8 | 6 mM | 0.6 mM | 25% glycerol + | 0.6% |
| | | | | | 25% propylene glycol | (w/v) |
| 6 | 1.0E+12 | 7.8 | 6 mM | 0.6 mM | 25% glycerol + | — |
| | | | | | 25% propylene glycol | |

TABLE 7
Titer Analysis And Stability Studies

| Time (month): | | | | |
|---|---|---|---|---|
| 0 | 6 | 9 | 12 | 16 |

Titer Analysis by AEX-HPLC [VP/mL]

| | | | | | |
|---|---|---|---|---|---|
| 1 | 1.16E+12 | 1.09E+12 | 1.23E+12 | 1.25E+12 | 1.14E+12 |
| 2 | 1.09E+12 | 1.05E+12 | 1.06E+12 | 1.15E+12 | 1.03E+12 |
| 3 | 1.12E+12 | 1.04E+12 | 1.16E+12 | 1.13E+12 | 1.16E+12 |
| 4 | 1.11E+12 | 1.04E+12 | 1.05E+12 | 1.13E+12 | 9.74E+11 |
| 5 | 1.18E+12 | 1.03E+12 | 1.00E+12 | 1.07E+12 | 1.02E+12 |
| 6 | 1.12E+12 | 1.02E+12 | 1.02E+12 | 1.04E+12 | 1.06E+12 |

Titer Analysis by AEX-HPLC (Percent Initial)

| | | | | | |
|---|---|---|---|---|---|
| 1 | 100% | 94% | 106% | 108% | 98% |
| 2 | 100% | 96% | 97% | 106% | 94% |
| 3 | 100% | 93% | 104% | 101% | 104% |
| 4 | 100% | 94% | 95% | 102% | 88% |
| 5 | 100% | 87% | 85% | 91% | 87% |
| 6 | 100% | 91% | 91% | 93% | 95% |

Shift in Retention Time Analysis by AEX-HPLC (minutes)

| | | | | | |
|---|---|---|---|---|---|
| 1 | −0.014 | 0.021 | 0.046 | 0.067 | 0.075 |
| 2 | −0.035 | −0.044 | −0.036 | −0.034 | −0.056 |
| 3 | −0.017 | 0.039 | 0.078 | 0.092 | 0.108 |
| 4 | −0.034 | −0.053 | −0.042 | −0.049 | −0.071 |
| 5 | −0.013 | −0.033 | −0.010 | −0.020 | −0.037 |
| 6 | −0.015 | −0.033 | −0.013 | −0.013 | −0.038 |

pVI Analysis by RP-HPLC (Percent Initial)

| | | | | | |
|---|---|---|---|---|---|
| 1 | 100% | 84% | 87% | 93% | 107% |
| 2 | 100% | 99% | 97% | 91% | 108% |
| 3 | 100% | 80% | 86% | 75% | 104% |
| 4 | 100% | 92% | 93% | 89% | 99% |
| 5 | 100% | 90% | 81% | 80% | 94% |
| 6 | 100% | 96% | 84% | 85% | 104% |
| Ref. | 100% | 105% | 87% | 89% | 94% |

GM-CSF Secretion Levels (Relative to the −70° C. CTL)

| | | | | | |
|---|---|---|---|---|---|
| 1 | 1.1 | 1.0 | 1.1 | 0.9 | 1.0 |
| 2 | 0.9 | 0.9 | 0.9 | 1.0 | 1.0 |
| 3 | 1.0 | 1.0 | 1.1 | 1.0 | 1.1 |
| 4 | 0.7 | 0.9 | 1.0 | 1.0 | 1.1 |
| 5 | 0.9 | 0.9 | 1.1 | 0.9 | 1.1 |
| 6 | 0.9 | 0.7 | 1.1 | 0.9 | 1.1 |
| R-15 | 1.0 | 1.0 | 1.0 | 1.0 | 1.0 |

Plaque (Pfu/ml)

| | | | | | |
|---|---|---|---|---|---|
| 1 | ND | ND | 8.8E+10 | 1.3E+11 | 5.5E+10 |
| 2 | ND | ND | 9.0E+10 | 1.2E+11 | 7.5E+10 |
| 3 | ND | ND | 8.0E+10 | 1.3E+11 | 3.6E+10 |
| 4 | ND | ND | 1.6E+11 | 1.3E+11 | 6.5E+10 |
| 5 | ND | ND | 9.3E+10 | 1.2E+11 | 5.8E+10 |
| 6 | ND | ND | 9.8E+10 | 1.4E+11 | 7.5E+10 |

A number of different cosolvent formulations (listed in Table 8) were used to test viral stocks containing 1E12 and 2E12 VP/mL at 5° C. for up to 20 months. Each cosolvent formulation was shown to exhibit enhanced stability of a stock of virus relative to the ARCA formulation. The results of titer analysis and stability studies for each formulation listed in Table 8 are provided in Table 9. Each formulation tested showed good stability for up to 20 months, as determined by an analysis of % ICV=Intact Capsid Virion; [ICV]=[VP] at time-zero; change in RT, plaque (VP/PFU) and GSR=GM-CSF Secretion Rate normalized to reference standard; wherein <#> represents the %[VP]o that are now Penton-Vacant Virions with no ICV present.

TABLE 8
Formulations Tested.

| | | Initial |
|---|---|---|
| | | Particle Titer |
| # | Formulation | (VP/mL) |
| H1 | ARCA | 1.8E+12 |
| H2 | ARCA + 1% Thioglycerol + 15DMS | 1.9E+12 |
| H5 | ARCA-P80 + 15% Sucrose | 1.8E+12 |
| H8 | ARCA-P80 + 30% Sucrose | 1.8E+12 |
| H11 | ARCA-P80-Sucrose + 20% Glycerol | 1.8E+12 |
| H18 | ARCA-P80-Sucrose + 15% P. Glycol | 1.9E+12 |
| H22 | ARCA-P80-Sucrose + 5% P. Glycol + 15 mM DMS | 1.8E+12 |
| M1 | ARCA | 9.2E+11 |
| M2 | ARCA + 1% Thioglycerol | 9.0E+11 |
| M4 | ARCA + 15 mM DMS | 8.9E+11 |
| M6 | ARCA-P80 + 15% Sucrose | 9.4E+11 |
| M8 | ARCA-P80 + 30% Sucrose | 9.1E+11 |
| M10 | ARCA-P80-Sucrose + 20% Glycerol | 9.6E+11 |
| M17 | ARCA-P80-Sucrose + 5% Glycerol + 15 mM DMS | 9.3E+11 |
| M20 | ARCA-P80-Sucrose + 10% P. Glycol | 9.1E+11 |
| M22 | ARCA-P80-Sucrose + 5% P. Glycol | 9.3E+11 |

ARCA = 5% Sucrose, 1% Glycine, 10 mM Tris, 1 mM Magnesium Chloride, RT-pH 7.8;
DMS = Dimethyl Sulfide;
P80 = Polysorbate-80;
P. Glycol = Propylene Glycol

TABLE 9A
CG0070 formulated in various formulations at 1E12 and 2E12 VP/mL and stored at 5° C. (2-8° C.)

| 0 Months | | | | | 1 Month | | | 4 Months | | | 6 Months | | |
|---|---|---|---|---|---|---|---|---|---|---|---|---|---|
| | % | Change | | | % | Change | | % | Change | | % | Change | |
| | [ICV]o | in RT | Plaque | | [ICV]o | in RT | | [ICV]o | in RT | | [ICV]o | in RT | |
| | % | (min) | VP/pfu | GSR | % | (min) | GSR | % | (min) | GSR | % | (min) | GSR |
| H1 | 100% | −0.025 | 8 | 1.0 | 100% | −0.002 | 0.8 | <50%> | 0.441 | 0.0 | <1% | 0.629 | 0.0 |
| H2 | 100% | −0.019 | 8 | 1.0 | 97% | −0.028 | 0.8 | 100% | 0.001 | 0.7 | 83% | 0.026 | 0.9 |
| H5 | 100% | −0.015 | 9 | 1.0 | 100% | 0.002 | 0.9 | 105% | 0.065 | 0.8 | 96% | 0.113 | 1.1 |
| H8 | 100% | −0.036 | 10 | 1.1 | 104% | −0.014 | 0.6 | 108% | 0.041 | 0.8 | 100% | 0.073 | 1.0 |
| H11 | 100% | −0.035 | 21 | 1.0 | 102% | −0.001 | 0.7 | 107% | 0.054 | 1.0 | 98% | 0.087 | 1.1 |
| H18 | 100% | −0.033 | 15 | 0.9 | 102% | −0.013 | 0.9 | 107% | 0.041 | 1.0 | 99% | 0.091 | 1.0 |
| H22 | 100% | −0.033 | 13 | 1.1 | 102% | −0.032 | 0.9 | 106% | 0.048 | 0.9 | 99% | 0.078 | 0.9 |
| M1 | 100% | −0.041 | 9 | 1.1 | ND | ND | ND | 94% | 0.073 | 0.8 | 79% | 0.227 | 0.2 |
| M2 | 100% | −0.038 | 9 | 1.1 | ND | ND | ND | 104% | 0.045 | 0.9 | 95% | 0.042 | 0.9 |
| M4 | 100% | −0.038 | 10 | 1.2 | ND | ND | ND | 97% | 0.068 | 0.8 | 94% | 0.115 | 0.9 |
| M6 | 100% | −0.034 | 8 | 1.0 | ND | ND | ND | 97% | 0.058 | 0.7 | 94% | 0.117 | 0.8 |
| M8 | 100% | −0.034 | 16 | 1.1 | ND | ND | ND | 100% | 0.033 | 0.5 | ND | ND | ND |
| M10 | 100% | −0.031 | 15 | 1.2 | ND | ND | ND | 101% | 0.037 | 0.7 | ND | ND | ND |
| M17 | 100% | −0.030 | 17 | 1.1 | ND | ND | ND | 101% | 0.020 | 0.8 | 96% | 0.081 | 0.9 |
| M20 | 100% | −0.036 | 12 | 1.0 | ND | ND | ND | 105% | 0.024 | 0.9 | 98% | 0.093 | 1.0 |
| M22 | 100% | −0.033 | 11 | 1.1 | ND | ND | ND | 104% | 0.015 | 0.7 | 98% | 0.089 | 0.9 |

TABLE 9B
CG0070 formulated in various formulations at 1E12 and 2E12 VP/mL and stored at 5° C. (2-8° C.)

| 9 Months | | | | | 12 Months | | | | 16 Months | | | | 20 Months | | | |
|---|---|---|---|---|---|---|---|---|---|---|---|---|---|---|---|---|
| | % | | | | % | | | | % | | | | % | | | |
| | [ICV]o | □ RT | Plaque | | [ICV]o | □ RT | Plaque | | [ICV]o | □ RT | Plaque | | [ICV]o | □ RT | Plaque | |
| | % | (min) | VP/pfu | GSR | % | (min) | VP/pfu | GSR | % | (min) | VP/pfu | GSR | % | (min) | VP/pfu | GSR |
| H1 | <1% | 0.677 | >1000 | 0.0 | <1% | 1.102 | ND | ND | <1% | 0.758 | ND | ND | <1% | 0.758 | ND | ND |
| H2 | 38% | 0.371 | >1000 | 0.0 | <1% | 0.827 | ND | <0.01 | <1% | 0.585 | ND | ND | <1% | 0.584 | ND | ND |
| H5 | 98% | 0.151 | 21 | 1.0 | 94% | 0.295 | 22 | 0.4 | 89% | 0.227 | 22 | 0.9 | 93% | 0.256 | 11 | 0.9 |
| H8 | 100% | 0.125 | 16 | 1.0 | 99% | 0.233 | 18 | 0.9 | 98% | 0.199 | 22 | 1.0 | 98% | 0.230 | 13 | 1.2 |
| H11 | 99% | 0.136 | 12 | 1.2 | 96% | 0.272 | 15 | 1.0 | 96% | 0.220 | 18 | 1.0 | 95% | 0.251 | 11 | 0.9 |
| H18 | ND | ND | ND | ND | 100% | 0.289 | 20 | 0.9 | 97% | 0.216 | 22 | 0.9 | 98% | 0.236 | 14 | 1.0 |
| H22 | 100% | 0.118 | 14 | 1.1 | 100% | 0.258 | 20 | 0.8 | 95% | 0.199 | 11 | 1.0 | 97% | 0.214 | 8 | 1.0 |
| M1 | <1% | 0.508 | >1000 | 0.0 | <1% | 0.948 | ND | <0.01 | <1% | 0.694 | >100 | <0.01 | <1% | 0.718 | ND | ND |
| M2 | 75% | 0.103 | 48 | 0.7 | 19% | 0.350 | 154 | 0.1 | <1% | 0.544 | >100 | 0.1 | <1% | 0.529 | ND | 0.01 |
| M4 | 92% | 0.165 | 14 | 0.9 | 82% | 0.325 | 24 | 0.7 | 56% | 0.290 | 22 | 0.5 | 46% | 0.333 | ND | 0.02 |
| M6 | 94% | 0.150 | 17 | 0.8 | 92% | 0.293 | 16 | 1.0 | 88% | 0.236 | 16 | 1.2 | 92% | 0.250 | 10 | 0.6 |
| M8 | 97% | 0.124 | 19 | 1.0 | 96% | 0.249 | 19 | 0.8 | 94% | 0.196 | 20 | 1.0 | 101% | 0.235 | 12 | 0.8 |
| M10 | 96% | 0.135 | 20 | 1.0 | 95% | 0.275 | 16 | 1.0 | 92% | 0.205 | 14 | 1.0 | 95% | 0.245 | 6 | 0.9 |
| M17 | 95% | 0.124 | 20 | 0.9 | 92% | 0.248 | 16 | 1.0 | 88% | 0.200 | 22 | 0.8 | 96% | 0.241 | 10 | 0.9 |
| M20 | 99% | 0.136 | 19 | 0.9 | 98% | 0.273 | 15 | 0.8 | 96% | 0.220 | 19 | 0.9 | 94% | 0.225 | 11 | 0.7 |
| M22 | 98% | 0.134 | 22 | 1.0 | 97% | 0.271 | 19 | 1.0 | 91% | 0.209 | 16 | 1.0 | 96% | 0.238 | 17 | 0.8 |

A number of different dimethyl sulfide and propylene glycol cosolvent formulations (listed in Table, 10) were used to test viral stocks containing 4E12 VP/mL at 5° C. for up to 4 months. Each cosolvent formulation was shown to exhibit enhanced stability of a stock of virus relative to the ARCA formulation. The results of titer analysis and stability studies for each formulation listed in Table 10 are provided in Table 11. Each formulation tested showed good stability for up to 4 months, as determined by an analysis of % ICV=Intact Capsid Virion; [ICV]=[VP] at time-zero; change in RT, plaque (VP/PFU) and GSR=GM-CSF Secretion Rate normalized to reference standard; wherein <#> represents the %[VP]o that are now Penton-Vacant Virions with no ICV present.

TABLE 10
Formulations Tested.

| | | Titer |
|---|---|---|
| # | Formulation | (VP/mL) |
| T75-1 | ARCA −70° C. | 4.3E+12 |
| T75-2 | ARCA | 4.1E+12 |
| T75-3 | ARCA + 15 mM DMS | 4.2E+12 |
| T75-9 | ARCA-P80-Sucrose + 10% PropGol | 4.0E+12 |
| T75-10 | ARCA-P80-Sucrose + 10% PropGol + 15 mM DMS | 4.0E+12 |
| T75-11 | ARCA-P80-Sucrose + 10% PropGol + 20 mM Met | 4.0E+12 |
| T75-12 | ARCA-P80-Sucrose + 5% PropGol | 4.0E+12 |
| T75-13 | ARCA-P80-Sucrose + 5% PropGol + 15 mM DMS | 4.0E+12 |
| T75-14 | ARCA-P80-Sucrose + 5% PropGol + 20 mM Met | 4.2E+12 |

ARCA = 5% Sucrose, 1% Glycine, 10 mM Tris, 1 mM Magnesium Chloride, RT-pH 7.8;
DMS = Dimethyl Sulfide;
P80 = Polysorbate-80;
PropGol = Propylene Glycol

TABLE 11
CG0070 formulated in various formulations at 4E12 VP/mL and stored at 5° C. (2-8° C.)

| | Time [month] | | | | | | | | | | | | | | | | |
|---|---|---|---|---|---|---|---|---|---|---|---|---|---|---|---|---|---|
| | 0 | | | | 1 | | | 1.5 | | | 2 | | | 4 | | | |
| | % | | | | % | | | % | | | % | | | % | | | |
| | [ICV]o | Δ RT | Plaque | | [ICV]o | Δ RT | | [ICV]o | Δ RT | | [ICV]o | D RT | | [ICV]o | Δ RT | Plaque | |
| # | % | (min) | VP/pfu | GSR | % | (min) | GSR | % | (min) | GSR | % | (min) | GSR | % | (min) | VP/pfu | GSR |
| T75-1 | 100% | 0.026 | 19 | 1.0 | 98% | 0.023 | 0.6 | ND | ND | ND | 97% | 0.026 | 1.0 | 96% | 0.020 | 10 | 1.1 |
| T75-2 | 100% | 0.020 | 11 | 1.0 | <54%> | 0.539 | <0.01 | ND | ND | ND | ND | ND | ND | ND | ND | ND | ND |
| T75-3 | 100% | 0.025 | 19 | 1.0 | 95% | 0.032 | 0.7 | 76% | 0.049 | 0.9 | <44%> | 0.534 | 0.3 | ND | ND | ND | ND |
| T75-9 | 100% | 0.018 | 22 | 1.0 | 101% | 0.034 | 1.3 | ND | ND | ND | 100% | 0.052 | 1.1 | 97% | 0.073 | 20 | 1.1 |
| T75-10 | 100% | 0.021 | 21 | 1.0 | 99% | 0.039 | 1.2 | ND | ND | ND | 99% | 0.056 | 1.1 | 96% | 0.072 | 21 | 1.1 |
| T75-11 | 100% | 0.014 | 17 | 1.0 | 99% | 0.029 | 1.2 | ND | ND | ND | 101% | 0.048 | 1.0 | 94% | 0.066 | 21 | 1.2 |
| T75-12 | 100% | 0.013 | 24 | 1.0 | 101% | 0.028 | 1.1 | 89% | 0.016 | 1.1 | 99% | 0.042 | 1.1 | 99% | 0.068 | 16 | 1.1 |
| T75-13 | 100% | 0.005 | 18 | 1.0 | 99% | 0.031 | 1.1 | 95% | 0.013 | 1.1 | 100% | 0.041 | 0.9 | 99% | 0.066 | 13 | 1.0 |
| T75-14 | 100% | 0.008 | 18 | 1.0 | 99% | 0.027 | 1.0 | 91% | 0.009 | 1.1 | 93% | 0.048 | 1.0 | 93% | 0.064 | 16 | 1.2 |

ICV = Intact Capsid Virion; [ICV] = [VP] at time-zero; <#> represents the % [VP]o that are now Penton-Vacant Virions with no ICV present
GSR = GM-CSF Secretion Rate normalized to reference standard
ND = Not Done

Enhanced Stability of Hexon in Formulations Containing ARCA and DMS

A study was carried out to further understand the mechanism by which thio compounds reversibly inhibit the adenoviral protease. In these studies a virus preparation was formulated in ARCA buffer plus DMS. A forced oxidation experiment showed hexon to be preferentially oxidized over protein VI, and that oxidation of hexon and protein VI did not impact biological functionality as determined by an assay for GM-CSF secretion rate. However, preferential degradation of protein VI over hexon was shown as a function of time when virus was stored at the enzymatically active temperature of 30° C. In this study degradation that correlated with biological functionality as determined by an assay for GM-CSF is inhibited by addition of DMS to the formulation.

In a related study, increasing concentrations of DMS (15 mM and 150 mM, respectively) were shown to correlate with less modification of hexon by oxidation, and the inclusion of 15 mM DMS in the ARCA formulation was shown to significantly inhibit hexon oxidation over a study duration of 20 months.

Although the foregoing invention has been described in some detail by way of illustration and example for purposes of clarity and understanding, it will be apparent to those skilled in the art that certain changes and modifications may be practiced. Various aspects of the invention have been achieved by a series of experiments, some of which are described by way of the following non-limiting examples. Therefore, the description and examples should not be construed as limiting the scope of the invention, which is delineated by the appended claims. All publications, patents, and patent applications cited herein are hereby incorporated by reference in their entirety for all purposes.

What is claimed is:

1. A formulation comprising an adenoviral vector, glycine, MgCl2, Tris buffer and an aqueous cosolvent selected from the group consisting of propylene glycol, DMSO, PEG, sucrose, glycerol and glycofurol wherein said formulation exhibits greater stability from 2° C. to 30° C. than a formulation lacking said aqueous cosolvent.

2. The formulation according to claim 1, wherein said aqueous cosolvent is propylene glycol.

3. The formulation according to claim 2, wherein said propylene glycol is present at a concentration of from about 3 to 20%.

4. The formulation according to claim 1, wherein said aqueous cosolvent is glycofurol.

5. The formulation according to claim 4, wherein said glycofurol is present at a concentration of from about 5 to 20%.

6. The formulation according to claim 1, wherein said aqueous cosolvent is sucrose.

7. The formulation according to claim 6, wherein said sucrose is present at a total concentration of at least 20%.

8. The formulation according to claim 1, wherein said aqueous cosolvent is glycerol.

9. The formulation according to claim 8, wherein said glycerol is present at a concentration of from about 10 to 50%.

10. The formulation according to claim 1, wherein said formulation is stored at about 5° C.

11. The formulation according to claim 1, wherein said formulation is stored at about room temperature (15-30° C.).

12. A formulation comprising a viral vector which relies on a viral encoded intracapsid protease for cell entry and a reversible protease inhibitor wherein said formulation exhibits greater stability from 2° C. to 30° C. than a formulation lacking said reversible protease inhibitor.

13. A formulation according to claim 12, wherein said viral encoded intracapsid protease is an adenoviral protease.

14. The formulation according to claim 13, wherein said reversible protease inhibitor is an inhibitor of an L3/p23 cysteine protease.

15. The formulation according to claim 14, wherein said reversible protease inhibitor is selected from the group consisting of thio compounds including thioglycerol, dimethyl sulfide, dithiothreitol (DTT), cysteine, glutathione, and methionine.

16. A formulation comprising an adenoviral vector, glycine, MgCl2, Tris buffer and a thio compound wherein said formulation exhibits greater stability from about 2° C. to 30° C. than a formulation lacking said thio compound.

17. A formulation according to claim 16, wherein said thio compound is thioglycerol.

18. The formulation according to claim 17, wherein said thioglycerol is present at a concentration of from about 0.5 to 2.0% or about 50 to 200 mM.

19. A formulation according to claim 16, wherein said thio compound is dimethyl sulfide.

20. The formulation according to claim 19, wherein said dimethyl sulfide is present at a concentration of from about 10 to 100 mM.

21. A formulation according to claim 16, wherein said thio compound is DTT.

22. The formulation according to claim 21, wherein said DTT is present at a concentration of from about 20-100 mM.

23. The formulation according to claim 22, wherein said DTT is present at a concentration of 50 mM.

24. A formulation according to claim 16, wherein said thio compound is cysteine.

25. The formulation according to claim 24, wherein said cysteine is present at a concentration of at least 1% or 150 mM.

26. The formulation according to claim 12, wherein said formulation is stored at 5° C.

27. The formulation according to claim 16, wherein said formulation is stored at 5° C.

28. The formulation according to claim 12, wherein said formulation is stored at room temperature (15-30° C.).

29. The formulation according to claim 16, wherein said formulation is stored at room temperature.

30. A formulation according to claim 12, wherein said formulation further comprises an aqueous cosolvent selected from the group consisting of propylene glycol, DMSO, PEG, sucrose, glycerol and glycofurol.

31. A formulation according to claim 16, wherein said formulation further comprises an aqueous cosolvent selected from the group consisting of propylene glycol, DMSO, PEG, sucrose, glycerol and glycofurol.

32. The formulation according to claim 31, wherein said aqueous cosolvent is propylene glycol.

33. The formulation according to claim 32, wherein said propylene glycol is present at a concentration of from about 3 to 20%.

34. The formulation according to claim 31, wherein said aqueous cosolvent is glycofurol.

35. The formulation according to claim 34, wherein said glycofurol is present at a concentration of from about 5 to 20%.

36. The formulation according to claim 31, wherein said aqueous cosolvent is sucrose.

37. The formulation according to claim 36, wherein said sucrose is present at a total concentration of at least 35%.

38. The formulation according to claim 31, wherein said aqueous cosolvent is glycerol.

39. The formulation according to claim 38, wherein said glycerol is present at a concentration of from about 10 to 50%.

40. The formulation according to claim 31, wherein said formulation is stored at 5° C.

41. The formulation according to claim 31, wherein said formulation is stored at room temperature (15-30° C.).
